(19) United States
(12) Patent Application Publication
(10) Pub. No.: US 2026/0048234 A1
(43) Pub. Date: Feb. 19, 2026

(54) CATHETERS WITH INTEGRATED STABILITY AND PLANARITY FEATURES

(71) Applicant: Boston Scientific Scimed, Inc., Maple Grove, MN (US)

(72) Inventors: Emily Rose Whitwam, Osseo, MN (US); Jason John Matteson, Beldenville, WI (US); Matthew Nelson Frost, New Brighton, MN (US); Chris Nazy, Bloomington, MN (US)

(21) Appl. No.: 19/301,295

(22) Filed: Aug. 15, 2025

Related U.S. Application Data
(60) Provisional application No. 63/684,046, filed on Aug. 16, 2024.

(51) Int. Cl. A61M 25/00; A61M 25/01

(52) U.S. Cl. CPC A61M 25/0053; A61M 25/0012; A61M 25/0136

(57) ABSTRACT

A medical catheter with enhance structural integration and performance is disclosed. The catheter features a flexible shaft with a proximal portion formed from a proximal shaft component and a distal portion formed from a distal shaft component. A transition region between the proximal and distal portions includes protrusions of the distal shaft component extending into the proximal shaft component, providing improved stability and planarity during use of the catheter. The protrusions are formed from a transition process between the proximal and distal components. The catheter may incorporate structural elements positioned within the flexible shaft in relation to the transition region to enhance the performance of the catheter. The disclosed design and manufacturing process results in a medical catheter with seamless integration of shaft components, optimized flexibility, and precise steering control, suitable for minimally invasive procedures.

### CROSS REFERENCE TO RELATED APPLICATIONS

This application claims priority to U.S. Provisional Patent Application No. 63/684,046 entitled “CATHETERS WITH INTEGRATED STABILITY AND PLANARITY FEATURES,” filed Aug. 16, 2024, which is herewith incorporated by reference in its entirety.

### TECHNICAL FIELD

The present invention relates to medical catheters and methods for manufacturing medical catheters. More specifically, the invention relates to medical catheters with enhanced structural integration and performance through unique shaft designs and manufacturing processes.

### BACKGROUND

Medical catheters are commonly used in minimally invasive medical procedures to navigate through complex anatomical pathways and access target sites within the body. These catheters typically include a flexible elongated shaft with a steerable distal portion that can be deflected or curved by the operator to facilitate navigation and positioning. The steering mechanism often involves the use of pull wires or other tensioning elements that extend through the catheter shaft and connect to the distal portion, allowing the operator to control the deflection of the catheter tip.

To achieve optimal performance and maneuverability, catheters rely on the integration of various structural elements within the catheter shaft, such as reinforcing braids, coils, or articulation structures. However, efficiently manufacturing these catheters with multiple materials and components can be challenging. Conventional methods often involve time-consuming and inconsistent manual processes, such as joining separate proximal and distal shaft portions using adhesives or heat bonding. These techniques may result in abrupt transitions in mechanical properties along the catheter shaft, potentially compromising the catheter's flexibility, pushability, and steering responsiveness.

Furthermore, the integration of additional structural elements, such as Bowden assemblies for steering wire guidance or articulation structures for controlled deflection, can be complex and may require specialized manufacturing steps.

The present invention addresses these challenges by providing improved devices and methods for manufacturing medical catheters with enhanced structural integration and performance. By utilizing different shaft designs and manufacturing techniques, such as reel-to-reel processing and selective material removal, the present invention enables the creation of seamless transitions between shaft components and the efficient integration of structural elements. The shaft design, which incorporates protrusions formed from the distal shaft component extending into the proximal shaft component, enhances the catheter's flexibility, pushability, and steering characteristics. This results in a catheter with optimized maneuverability and effectiveness in minimally invasive procedures.

### SUMMARY

In Example 1, a medical catheter comprises a handle configured for manipulation by a user; a flexible shaft having a proximal portion, a distal portion, and a transition region between the proximal and distal portions of the shaft wherein: the proximal portion of the shaft extends distally from the handle and is formed from a proximal shaft component wherein a first length of the proximal shaft component extends distally; wherein distal portion of the shaft is formed from a distal shaft component wherein a second length of the distal shaft component extends proximally, and wherein the transition region of the shaft comprises the first length of the proximal shaft component and the second length of the distal shaft component.

In Example 2, the medical catheter of Example 1, wherein the second length of the distal portion includes at least two protrusions formed from the distal shaft component, the at least two protrusions being circumferentially spaced apart and extending longitudinally from the second length of the distal portion into the first length of the proximal portion.

In Example 3, the medical catheter of Example 2, wherein the at least two protrusions are formed as a result of a transition process between proximal shaft component and the distal shaft component.

In Example 4, the medical catheter of Example 3, wherein the transition process comprises removing portions of the proximal shaft component to expose a proximal reinforcing braid by a removal process, wherein at least two recesses are formed of the proximal reinforcing braid on the proximal shaft component from the removal process.

In Example 5, the medical catheter of any of Examples 2-4, wherein the distal shaft component is disposed over the proximal shaft component such that the distal shaft component fills the at least two recesses to form the at least two protrusions extending longitudinally from the second length of the distal portion into the first length of the proximal portion.

In Example 6, the medical catheter of any of Examples 4-5, wherein the at least two recesses are circumferentially spaced apart and extending longitudinally along the first length of the proximal portion.

In Example 7, the medical catheter of any of Examples 3-6, wherein the transition process comprises a reel-to-reel process or of similar process.

In Example 8, the medical catheter of any of Examples 3-7, wherein the removal process comprises one or more techniques selected from the group consisting of laser ablation, mechanical cutting, chemical etching, thermal ablation, and other suitable removal processes.

In Example 9, the medical catheter of any of Examples 1-8, wherein the proximal portion comprises: an outer tubular jacket; a Bowden assembly disposed within the outer tubular jacket, the Bowden assembly comprising: a pair of diametrically opposed helical Bowden cables; wherein the Bowden cables establish reinforced passageways configured to provide lateral support for one or more steering elements extending from the proximal region to the distal region; a Bowden ring attached to the distal end of the Bowden cables, the Bowden ring comprising a ring structure having a groove defined around its circumference; wherein the Bowden ring includes passageways for the one or more steering elements extending from the proximal region to the distal region; and wherein tension applied to the one or more steering elements causes deflection at a deflectable region.

In Example 10, the medical catheter of Example 9, wherein the Bowden cables establish reinforced passageways configured to provide radial and lateral support for the one or more steering elements extending from the proximal region to the distal region.

In Example 11, the medical catheter of any of Examples 9-10, wherein the Bowden ring includes passageways for the one or more steering elements extending from the proximal region to the distal region.

In Example 12, the medical catheter of any of Examples 1-11, wherein the distal portion comprises: an outer tubular jacket; an articulation structure disposed within the outer tubular jacket, the articulation structure comprising a plurality of longitudinally-arranged articulation elements, one or more steering wire lumens, wherein the steering wire lumens extend through the articulation structure, and one or more reinforcing members extending through the articulation elements.

In Example 13, the medical catheter of Example 12, wherein the articulation elements comprises a plurality of tubular segments, a plurality of connecting segments, a plurality of articulation links, a plurality of joints, a plurality of reinforcing members, or combinations thereof.

In Example 14, the medical catheter of any of Examples 12-13, wherein the distal portion comprises a deflectable region defining a longitudinal axis.

In Example 15, the medical catheter of any of Examples 12-14, further comprising one or more steering wire lumens within the articulation elements and configured to receive respective steering elements for applying the deflection force, wherein the one or more steering wire lumens are each circumferentially offset from the one or more reinforcing members by about 90 degrees.

In Example 16, a medical catheter comprising: a flexible shaft having a proximal portion, a distal portion, and a transition region between the proximal and distal portions of the shaft wherein: the proximal portion of the shaft extends distally from the handle and is formed from a proximal shaft component wherein a first length of the proximal shaft component extends distally; wherein distal portion of the shaft is formed from a distal shaft component wherein a second length of the distal shaft component extends proximally, and wherein the transition region of the shaft comprises the first length of the proximal shaft component and the second length of the distal shaft component; and a structural element positioned within the shaft.

In Example 17, the medical catheter of Example 16, wherein the second length of the distal portion includes at least two protrusions formed from the distal shaft component, the at least two protrusions being circumferentially spaced apart and extending longitudinally from the second length of the distal portion into the first length of the proximal portion.

In Example 18, the medical catheter of Example 17, wherein the at least two protrusions are formed as a result of a transition process between proximal shaft component and the distal shaft component.

In Example 19, the medical catheter of Example 18, wherein the transition process comprises removing portions of the proximal shaft component to expose a proximal reinforcing braid by a removal process, wherein at least two recesses are formed of the proximal reinforcing braid on the proximal shaft component from the removal process.

In Example 20, the medical catheter of Example 19, wherein the distal shaft component is disposed over the proximal shaft components such that the distal shaft component fills the at least two recesses to form the at least two protrusions extending longitudinally from the second length of the distal portion into the first length of the proximal portion.

In Example 21, the medical catheter of Example 19, wherein the at least two recesses are circumferentially spaced apart and extending longitudinally along the first length of the proximal portion.

In Example 22, the medical catheter of Example 18, wherein the transition process comprises a reel-to-reel process or of similar process.

In Example 23, the medical catheter of Example 19, wherein the removal process comprises one or more techniques selected from the group consisting of laser ablation, mechanical cutting, chemical etching, thermal ablation, and other suitable removal processes.

In Example 24, the medical catheter of Example 16, wherein the structural element is a Bowden assembly, the Bowden assembly is positioned within the proximal portion of the shaft, wherein the Bowden assembly comprises: a pair of diametrically opposed helical Bowden cables, and a Bowden ring attached to the distal end of the Bowden cables, wherein the Bowden cables establish passageways configured for one or more steering elements extending from the proximal region to the distal region.

In Example 25, the medical catheter of Example 24, wherein the Bowden cables establish reinforced passageways configured to provide radial and lateral support for the one or more steering elements extending from the proximal region to the distal region.

In Example 26, the medical catheter of Example 24, wherein the Bowden ring includes passageways for the one or more steering elements extending from the proximal region to the distal region.

In Example 27, the medical catheter of Example 16, wherein the structural element is an articulation structure having a proximal end and is positioned within the distal portion of the shaft, the articulation structure comprising: a plurality of longitudinally-arranged articulation elements, and one or more reinforcing members extending through the articulation elements.

In Example 28, the medical catheter of Example 27, wherein the articulation elements comprises a plurality of tubular segments, a plurality of connecting segments, a plurality of articulation links, a plurality of joints, a plurality of reinforcing members, or combinations thereof.

In Example 29, the medical catheter of Example 28, further comprising one or more steering wire lumens within the articulation elements and configured to receive respective steering elements for applying a deflection force, wherein the one or more steering wire lumens are each circumferentially offset from the one or more reinforcing members by about 90 degrees.

In Example 30, the medical catheter of Example 29, wherein the distal portion comprises a deflectable region defining a longitudinal axis.

In Example 31, a medical catheter comprising: a flexible shaft having a proximal portion, a distal portion, and a transition region between the proximal and distal portions of the shaft wherein: the proximal portion of the shaft extends distally from the handle and is formed from a proximal shaft component wherein a first length of the proximal shaft component extends distally; wherein distal portion of the shaft is formed from a distal shaft component wherein a second length of the distal shaft component extends proximally, wherein the distal portion comprises a deflectable region defining a longitudinal axis, wherein the deflection region is configured to assume a curved shape when a deflection force is applied, and wherein the transition region of the shaft comprises the first length of the proximal shaft component and the second length of the distal shaft component; and a structural element positioned within the shaft.

In Example 32, the medical catheter of Example 31, wherein the structural element is a Bowden assembly, the Bowden assembly is positioned within the proximal portion of the shaft, wherein the Bowden assembly comprises: a pair of diametrically opposed helical Bowden cables, and a Bowden ring attached to the distal end of the Bowden cables, wherein the Bowden cables establish passageways configured for one or more steering elements extending from the proximal region to the distal region.

In Example 33, the medical catheter of Example 31, wherein the structural element is an articulation structure having a proximal end and is positioned within the distal portion of the shaft, the articulation structure comprising: The medical catheter of claim 31, wherein the structural element is an articulation structure having a proximal end and is positioned within the distal portion of the shaft, the articulation structure comprising: a pair of diametrically opposed helical Bowden cables, and a Bowden ring attached to the distal end of the Bowden cables, wherein the Bowden cables establish passageways configured for one or more steering elements extending from the proximal region to the distal region.

In Example 34, a method of producing an integrated medical catheter comprising: providing a proximal portion of a shaft, wherein the proximal portion is formed from a proximal shaft component wherein a first length of the proximal shaft component extends distally, providing a distal portion of a shaft, wherein the distal portion is form from a distal shaft component wherein a second length of the distal shaft component extends proximally, performing a transition process between the proximal shaft component and the distal shaft component to create a flexible shaft, positioning a structural element within the shaft, and securing a handle to the proximal portion of the catheter shaft, the handle configured for manipulation by a user.

In Example 35, the method of Example 34, wherein the transition process comprises: forming at least two recesses on the proximal shaft component using a removal process, the at least two recesses being circumferentially spaced apart and extending longitudinally along the first length of the proximal shaft component, and disposing the distal shaft component over the proximal shaft component such that the distal shaft component fills the at least two recesses to form at least two protrusions extending longitudinally from the second length of the distal shaft component into the first length of the proximal shaft component.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is an illustration of an example medical catheter, consistent with various aspects of the present disclosure.

FIG. 2A is an illustration of the medical catheter, shown in FIG. 1, as deflected in a first direction, consistent with various aspects of the present disclosure.

FIG. 2B is an illustration of the medical catheter, shown in FIG. 1, as deflected in a second direction, consistent with various aspects of the present disclosure.

FIG. 3A is a detailed illustration of a portion of a side-view illustration of the medical catheter, consistent with various aspects of the present disclosure.

FIG. 3B is a detailed illustration of a transition region of the medical catheter, consistent with various aspects of the present disclosure.

FIG. 4 is an illustration of an articulation structure and Bowden assembly with a Bowden ring positioned in the medical catheter, consistent with various aspects of the present disclosure.

While the disclosure is amenable to various modifications and alternative forms, specific embodiments have been shown by way of example in the drawings and are described in detail below. The intention, however, is not to limit the disclosure to the particular embodiments described. On the contrary, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) the features in a given example used across all examples. Thus, no one figure should be interpreted as having any dependency or requirement related to any single component or combination of components illustrated therein. Additionally, various components depicted in a given figure may be, in examples, integrated with various ones of the other components depicted therein (and/or components not illustrated), all of which are considered to be within the ambit of the present disclosure.

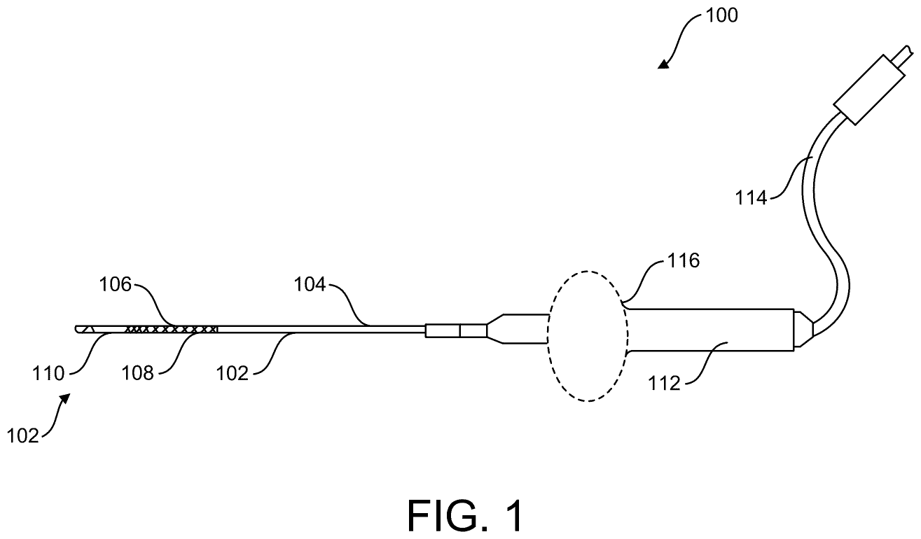

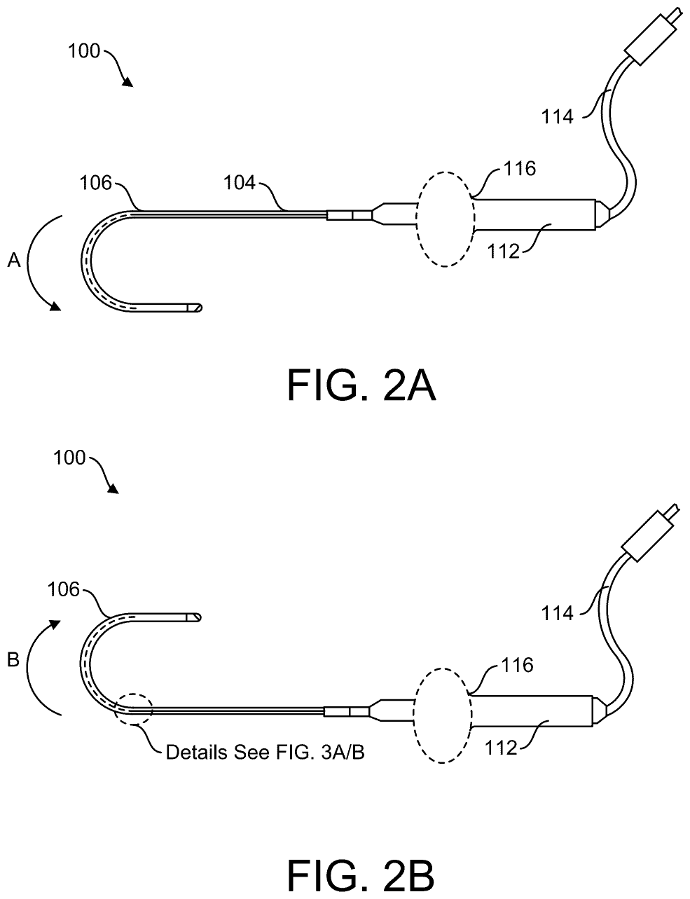

FIG. 1, FIGS. 2A and 2B are illustrations of an example medical catheter 100, consistent with various aspects of the present disclosure. As shown in FIG. 1, the medical catheter 100 may be a steerable medical catheter 100. In certain instances, the medical catheter 100 is steerable in one direction (e.g., direction A as shown in FIG. 2A) or in another direction (e.g., direction B as shown in FIG. 2B). The medical catheter 100 generally includes a tubular shaft 102 having a proximal portion 104 and a distal portion 106 that is sized and configured for placement and manipulation within in a target area of a patient. The distal portion 106 may be steerable. As shown, the distal portion 106 further includes a deflection region 108 and a distal end 110.

In certain instances, the tubular shaft 102 extends from a distal portion of a handle 112. An electrical cable or other suitable connector 114 extending from a proximal end of the handle 112 may be coupled to a source of energy or other equipment (not shown in FIG. 1) for transmitting one or more ablation signals. A steering actuator 116, such as a rotatable knob or plunger that may be arranged at the handle 112, may be manipulated by a physician to deflect or position the steerable distal portion 106 of the tubular shaft 102.

As shown in FIGS. 2A and 2B, the medical catheter 100 is of the deflectable or steerable type, such that during use, the deflection region 108 can be deflected or curved by a user to facilitate locating the distal end 110 at a desired target location within the heart. In embodiments, deflection of the deflection region 108 can be accomplished by manipulation of the steering actuator 118, which is operatively connected to steering elements (e.g., wires, tendons, ribbons, and the like) extending within and attached (directly or indirectly) to the shaft 102 at a location within the distal portion 106. The particular mode and structure for deflecting the deflection region 108 is not critical to the present disclosure, and so any technique, whether now known or later developed, can be employed within the scope of the present disclosure.

In use, deflecting or curving the distal portion 106 may impart a torsional force that could torque or twist the distal portion 106 away from or out of the target location. The deflection region 108 may torque out of plane from the plane in which the distal portion 106 was arranged prior to deflection due to the tension on the curvature of the medical catheter 100 within vasculature.

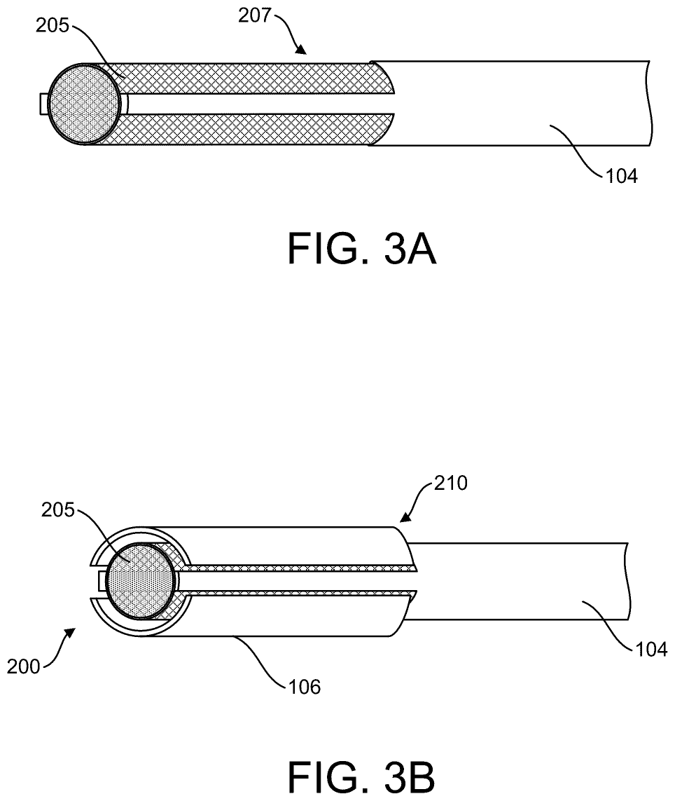

FIGS. 3A and 3B is an enlarged detail view of the medical catheter 100 illustrating the transition region 200 (FIG. 3B) between the proximal portion 104 and the distal portion 106 of the catheter shaft 102. The catheter shaft 102 comprises a proximal shaft component 208 and a distal shaft component 206.

In some embodiment, the transition region 200 is created through a reel-to-reel process, wherein the proximal shaft component 208 and the distal shaft component 206 are merged together. During this process, the distal shaft component 206 is introduced onto the proximal shaft component 208, resulting in the formation of at least two protrusions or “peaks” 210 of the distal shaft component 206 extending longitudinally into the proximal shaft component 208. These protrusions 210 are circumferentially spaced apart, typically at 180 degrees from each other, and serve to enhance the stability and planarity of the catheter shaft 102 during steering and navigation.

In another embodiment, the transition region 200 is created through a selective material removal process. Prior to the introduction of the distal shaft component 206, portions of the proximal shaft component 208 are mechanically etched away to create recesses 207 (FIG. 3A). This selective removal process exposes the underlying reinforcing braid 205 of the proximal shaft component 208 at specific locations where the recesses 207 are formed. The skilled artisan will recognize that the use of jackets in catheter construction is well known, and the particular details of the braid/jacket construction are not of critical importance to the present disclosure. In embodiments, the aforementioned braid can be omitted, or alternatively, other constructions, e.g., reinforcing coils, can be employed to enhance the structural and torsional strength of the jacket.

The selective removal of the proximal shaft component 208 can be achieved through various techniques, such as laser ablation, mechanical cutting, chemical etching, or thermal ablation. For example, laser ablation may be used to precisely remove portions of the proximal shaft component 208, creating well-defined recesses 207 with controlled depth, width, and shape. The laser parameters, such as wavelength, power, pulse duration, and spot size, can be adjusted to effectively remove the material without damaging the underlying reinforcing braid 205.

Once the recesses 207 are formed, the distal shaft component 206 is disposed over the proximal shaft component 208. As the distal shaft component 206 is introduced, it fills the recesses 207 and forms protrusions 210 that extend longitudinally from the distal portion 106 into the proximal portion 104. The protrusions 210 are securely anchored within the recesses 207, creating a smooth and continuous transition between the proximal shaft component 208 and the distal shaft component 206.

In some embodiments, the protrusions 210 and the exposed reinforcing braid 205 within the recesses 207 may be mechanically connected to further enhance the structural integrity of the transition region 200. This mechanical connection can be achieved through various methods, such as welding, soldering, adhesive bonding, or mechanical interlocking. For example, a compatible polymeric material may be used to fill the recesses 207 and bond the protrusions 210 to the exposed reinforcing braid 205, creating a seamless connection between the proximal shaft component 208 and the distal shaft component 206.

The combination of the reel-to-reel process and the selective material removal process allows for the creation of a shaft design with enhanced stability and planarity. The protrusions 210 extend into the proximal shaft component 208, providing additional support and resistance to torsional forces during steering and navigation. The exposed reinforcing braid 205 within the recesses 207 serves as an anchoring point for the protrusions 210, ensuring a secure and durable connection between the proximal and distal shaft components.

Furthermore, the selective removal process enables the customization of the transition region 200 based on specific catheter requirements. The number, size, shape, and location of the recesses 207 and protrusions 210 can be tailored to optimize the catheter's performance characteristics. For example, additional recesses and protrusions may be created to provide enhanced stability in certain regions of the catheter shaft 102, or the shape of the recesses may be modified to improve the flexibility and maneuverability of the transition region 200.

In some embodiments, a structural element may be positioned within the catheter shaft 102 to enhance the performance and functionality of the medical catheter 100. The structural element may extend from the proximal portion 104 to the distal portion 106 of the catheter shaft 102, passing through the transition region 200. The structural elements may be but are not limited to, a Bowden assembly or an articulation structure.

In some embodiments, the transition region 200 may comprise a single protrusion 210 of the distal shaft component 206 extending into the proximal shaft component 208, rather than the previously described configuration with at least two protrusions. To accommodate the single protrusion 210, a corresponding recess 207 is created on the proximal shaft component 208 through the selective material removal process. The recess 207 is formed at a location that matches the intended position of the protrusion 210, allowing for a secure and seamless integration between the proximal and distal shaft components. The selective removal process, such as laser ablation, is used to precisely remove a portion of the proximal shaft component 208, exposing the underlying reinforcing braid 205 at the desired location. The distal shaft component 206 may be disposed over the proximal shaft component 208, as the distal shaft component 206 is introduced, it fills the recess 207 and forms the single protrusion 210 that extends into the proximal portion 104. The protrusion 210 is securely anchored within the recess 207, creating a smooth and continuous transition between the proximal shaft component 208 and the distal shaft component 206.

The use of a single protrusion 210 enables for a more localized reinforcement of the transition region 200. By strategically positioning the protrusion 210 at a specific location, such as the point of maximum stress or deflection, the catheter shaft 102 can be tailored to withstand specific mechanical challenges. Further, the single protrusion 210 design provides greater flexibility in terms of catheter customization. The location and size of the protrusion 210 can be easily modified to suit different catheter specifications, enabling the development of specialized catheters for specific clinical applications.

Furthermore, the single protrusion 210 configuration may simplify the manufacturing process, as it requires the creation of only one recess 207 on the proximal shaft component 208. This can lead to reduced production time and cost, while still providing the necessary structural integration and performance enhancement.

In embodiments where a single protrusion 210 is utilized, the placement of the structural elements may be adjusted accordingly.

The shaft design and manufacturing process described above result in a medical catheter 100 with improved performance and maneuverability. The combination of the reel-to-reel process and the selective material removal process allows for the creation of a smooth and continuous transition between the proximal shaft component 208 and the distal shaft component 206, while the protrusions 210 and the exposed reinforcing braid 205 within the recesses 207 provide enhanced stability and planarity during steering and navigation. This innovative approach to catheter shaft design and manufacturing enables the development of advanced steerable catheters suitable for a wide range of minimally invasive medical procedures.

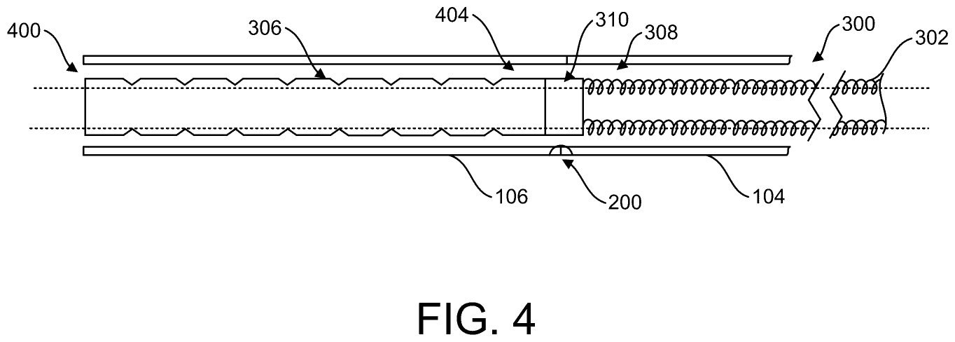

Referring now to FIG. 4, a Bowden assembly 300 is introduced within the medical catheter 100. The Bowden assembly 300, is a structural element within the medical catheter that facilitates steering control and maneuverability with the integration of Bowden cables 302 which may act as a reinforced steering wire guidance system within the catheter shaft, allowing for controlled deflection of the distal portion 106 while providing support and stability to the proximal portion 104. The Bowden assembly 300 is positioned within the proximal portion 104 of the medical catheter shaft 102 and extends into the distal portion 106 and comprises Bowden cables 302 and a Bowden ring 304, wherein the Bowden ring 304 may be composed from a metallic material or of a similar alloy. The Bowden cables 302 may comprise a pair of diametrically opposed helical coils that extends longitudinally through the proximal portion 104 of the shaft 102. Further, the Bowden cables 302 create dedicated passageways for one or more steering elements (e.g., wires, tendons, ribbons, and the like) 306. The one or more steering elements 306 are located in diametrically opposite positions and are operatively connected to the steering actuator 118 (FIG. 1) to facilitate the deflection of at least the deflection region 108 of the shaft 102 as known in the conventional manner. Additionally, the Bowden cables 302 may provide radial and lateral support and guidance for the one or more steering elements 306 by encasing them within the helical coils. The protective structure may help minimize the impact of any compressive or any other forces that may occur during catheter manipulation allowing the steering elements to maintain their integrity and functionally for consistent and precise deflection of the deflection region 108.

Further, at a distal end of the Bowden assembly 308, the Bowden cables 302 are securely attached to the Bowden ring 304, wherein the Bowden ring 304 includes passageways for the one or more steering elements 306 extending from the proximal region to the distal region. The Bowden ring is shown with a smooth, non-grooved Bowden ring 304, wherein the Bowden ring may be positioned underneath and at center of, or in close proximity to the attachment location 200 of the catheter shaft 102 serving as an anchoring point. The Bowden ring 304 may be connected to the shaft 102 by a mechanical connection. The mechanical connection may be done through a welding process, soldering process, adhesive process, polymer reflow process, or other processes that may allow and enhance the connection between the Bowden ring 304 and the exposed underlying reinforcing braid 205. The secure attachment of the Bowden ring to the shaft allows for a seamless transfer of steering forces from the proximal portion 104 to the distal portion 106, allowing control and maneuverability of the medical catheter 100.

In some embodiments, the Bowden ring 304 of the Bowden assembly 300 may be strategically placed at the location where the protrusions 210 of the distal shaft component 206 end. As shown in FIG. 3, the protrusions 210 extend from the distal portion 106 into the proximal portion 104, where in the protrusions 210 may reach up to the start of the deflection region 108. By positioning the Bowden ring at this location, the transition from the proximal shaft component 208 to the distal shaft component 206 is seamlessly integrated with the Bowden assembly. The Bowden cables extend proximally from the Bowden ring, through the proximal portion 104 of the catheter shaft 102 and terminate at the steering handle 112. This configuration allows for the efficient transmission of steering forces from the handle 112 to the deflection region 108, enabling precise control and maneuverability of the catheter tip.

In an example embodiment, the Bowden ring may be illustrated with a grooved Bowden ring. The grooved Bowden ring may feature a circumferential groove, wherein the circumferential groove may accommodate an adhesive. The circumferential groove allows an adhesive to fill the space, creating a connection between the Bowden ring and the shaft 102. The adhesive may flow through the braided gap 205 and flow into the groove, filling the voids and forming a strong bond that prevents slippage and maintains the integrity of the Bowden assembly 300. The choice between a grooved Bowden ring and the smooth Bowden ring 304 (FIG. 5) may depend on various factors, including the desired level of attachment strength, manufacturing feasibility, and specific requirements of the medical catheter application.

The strategic positioning and mechanical connection of the Bowden ring 304 to the shaft allows transfer of steering forces from the proximal portion 104 to the distal portion 106. The mechanical connection may help with overall control and maneuverability of the medical catheter 100 while maintaining a smooth transition between the proximal portion and distal portion without compromising the catheter's flexibility or introducing abrupt changes in stiffness enabling precise navigation through challenging anatomical structures.

In some embodiments, the Bowden assembly 300 may be adapted to suit specific catheter designs and applications. Variations in the number, configuration, and/or material composition of the Bowden cables 302 and Bowden ring 304 may be employed to optimize performance characteristics. For example, the helical coils of the Bowden cables may be designed with different diameters and materials, or the Bowden ring may be constructed of various materials to achieve the desired strength, and flexibility.

Further, FIG. 4 introduces an articulation structure 400 that may be positioned within the distal portion of the medical catheter shaft 102 and facilitates predictable, highly planar deflection of the deflection region 108 by resisting torsional forces on the shaft 102 that would otherwise tend to cause the deflection region 108 to deflect. The articulation structure 400 may comprise series of interconnected plurality of longitudinally-arranged articulation elements that work together to achieve the desired deflection characteristics. These articulation elements may comprise a plurality of longitudinally arranged tubular segments, a plurality of connecting segments, plurality of articulation links, a plurality of joints, a plurality of reinforcing members, or combinations thereof. The articulation structure 400 may include one or more reinforcing members embedded within the articulation elements and extending through the articulation structure 400 wherein the reinforcing members may maintain a curved shape of the deflection region 108 during deflection and maintain repeatability of achieving the curved shape of the deflection region 108 during deflection.

In an example embodiment the one or more reinforcing members may comprise a helically wound flat ribbon wire or stranded wire cables or of similar elements. The articulation structure 400 may further comprise one or more steering wire lumens arranged in parallel to the passageways 320, 322 of the Bowden assembly 300 wherein the steering wire lumens may be configured to receive one or more steering elements 306 that are operatively connected to the steering actuator 118 (FIG. 1) to facilitate the deflection of at least the deflection region 108 of the tubular shaft 102 as known in the conventional manner. The one or more steering wire lumens may also be circumferentially offset from the one or more reinforcing members by about 90 degrees. The specific design and arrangement of these elements can vary depending on the desired deflection profile and the intended application of the medical catheter 100.

As shown, the articulation structure 400 may be positioned adjacent or in close proximity to the Bowden assembly 300 wherein the distal end of the Bowden assembly 308 is in contact or near the proximal end of the articulation structure 404. This placement allows transfer of steering forces from the Bowden assembly 300 to the articulation structure 400, allowing smooth and controlled deflection of the distal portion of the medical catheter shaft 102. In response to a deflection force, the articulation structure 400 may bend in a direction A (FIG. 2A). In certain embodiments, the articulation structure 400 may bend in another direction B (FIG. 2B), opposite to that of direction A. Whether the articulation structure 400 is unidirectional or bidirectional is not of critical importance to the current invention.

The articulation structure 400 being arranged within the deflection region 108 of the tubular shaft 102 operates to stabilize at least the distal portion 106 of the tubular shaft 102. In other instances, the articulation structure 400 may extend along a length of the tubular shaft 102 or extend into an intermediate section between the proximal portion 104 and the distal portion 106.

The articulation structure 400 embedded within the deflection region 108 of the tubular shaft 102 may be caused to assume a curved shape when the tubular shaft 102 is arranged within a patient's vasculature. The ability to selectively curve the deflection region 108 can facilitate accurate and effective delivery therapy (e.g., ablation) or map the surface of the heart tissue (e.g., identify the locations of heart tissue that are a source of the arrhythmias).

In some embodiments, the articulation structure 400 may include a center lumen wherein the center lumen may include components such as one or more wires for ablation electrodes arranged along the shaft 102, navigational components, a temperature sensor (e.g., thermocouple), a force sensor, radio-frequency circuitry and/or wires, and a cooling lumen. The center lumen may be a working channel through which one or more devices may be passed.

In some embodiments, the proximal end of the articulation structure as may include a metallic component or of similar alloy, such as a ring or collar, designed to support the articulation structure and allow for mechanical connection to the catheter shaft. This metallic component may facilitate a smooth mechanical connection and provide additional strength and durability to the connection, allowing a stable integration of the articulation structure with the catheter shaft.

The placement of the Bowden assembly 300 and articulation structure 400, can be tailored based on the specific design requirements of the catheter 100. Factors such as the desired deflection profile, the length of the distal portion 106, and the overall catheter stiffness can influence the optimal placement of these components. As such, a user may strategically remove the polymeric material partially or fully by laser ablation or of similar removal process such that length, width, and depth of the exposed portion of the reinforcing braid 205 may be tailored to a desired size accommodate the integration of different structural elements to the shaft. Moreover, the pattern and arrangement of the exposed reinforcing braids 205 can be modified to achieve specific performance characteristics to contribute to the overall performance, maneuverability, and stability of the steerable medical catheter 100.

It is well understood that methods that include one or more steps, the order listed is not a limitation of the claim unless there are explicit or implicit statements to the contrary in the specification or claim itself. It is also well settled that the illustrated methods are just some examples of many examples disclosed, and certain steps may be added or omitted without departing from the scope of this disclosure. Such steps may include incorporating devices, systems, or methods or components thereof as well as what is well understood, routine, and conventional in the art.

The connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements. The scope is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B or C may be present in a single embodiment; for example, A and B, A and C, B and C, or A and B and C. The terms “couples,” “coupled,” “connected,” “attached,” and the like along with variations thereof are used to include both arrangements wherein two or more components are in direct physical contact and arrangements wherein the two or more components are not in direct contact with each other (e.g., the components are “coupled” via at least a third component), but still cooperate or interact with each other.

In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art with the benefit of the present disclosure to affect such feature, structure, or characteristic in connection with other embodiments whether or not explicitly described. After reading the description, it will be apparent to one skilled in the relevant art(s) how to implement the disclosure in alternative embodiments.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

We claim:

1. A medical catheter comprising:
a flexible shaft having a proximal portion, a distal portion, and a transition region between the proximal and distal portions of the shaft wherein:
the proximal portion of the shaft extends distally from the handle and is formed from a proximal shaft component wherein a first length of the proximal shaft component extends distally;
wherein distal portion of the shaft is formed from a distal shaft component wherein a second length of the distal shaft component extends proximally, and
wherein the transition region of the shaft comprises the first length of the proximal shaft component and the second length of the distal shaft component; and
a structural element positioned within the shaft.

2. The medical catheter of claim 1, wherein the second length of the distal portion includes at least two protrusions formed from the distal shaft component, the at least two protrusions being circumferentially spaced apart and extending longitudinally from the second length of the distal portion into the first length of the proximal portion.

3. The medical catheter of claim 2, wherein the at least two protrusions are formed as a result of a transition process between proximal shaft component and the distal shaft component.

4. The medical catheter of claim 3, wherein the transition process comprises removing portions of the proximal shaft component to expose a proximal reinforcing braid by a removal process, wherein at least two recesses are formed of the proximal reinforcing braid on the proximal shaft component from the removal process.

5. The medical catheter of claim 4, wherein the distal shaft component is disposed over the proximal shaft components such that the distal shaft component fills the at least two recesses to form the at least two protrusions extending longitudinally from the second length of the distal portion into the first length of the proximal portion.

6. The medical catheter of claim 4, wherein the at least two recesses are circumferentially spaced apart and extending longitudinally along the first length of the proximal portion.

7. The medical catheter of claim 3, wherein the transition process comprises a reel-to-reel process or of similar process.

8. The medical catheter of claim 4, wherein the removal process comprises one or more techniques selected from the group consisting of laser ablation, mechanical cutting, chemical etching, thermal ablation, and other suitable removal processes.

9. The medical catheter of claim 1, wherein the structural element is a Bowden assembly, the Bowden assembly is positioned within the proximal portion of the shaft, wherein the Bowden assembly comprises:
a pair of diametrically opposed helical Bowden cables, and a Bowden ring attached to the distal end of the Bowden cables, wherein the Bowden cables establish passageways configured for one or more steering elements extending from the proximal region to the distal region.

10. The medical catheter of claim 9, wherein the Bowden cables establish reinforced passageways configured to provide radial and lateral support for the one or more steering elements extending from the proximal region to the distal region.

11. The medical catheter of claim 9, wherein the Bowden ring includes passageways for the one or more steering elements extending from the proximal region to the distal region.

12. The medical catheter of claim 1, wherein the structural element is an articulation structure having a proximal end and is positioned within the distal portion of the shaft, the articulation structure comprising:
a plurality of longitudinally-arranged articulation elements, and
one or more reinforcing members extending through the articulation elements.

13. The medical catheter of claim 12, wherein the articulation elements comprises a plurality of tubular segments, a plurality of connecting segments, a plurality of articulation links, a plurality of joints, a plurality of reinforcing members, or combinations thereof.

14. The medical catheter of claim 13, further comprising one or more steering wire lumens within the articulation elements and configured to receive respective steering elements for applying a deflection force, wherein the one or more steering wire lumens are each circumferentially offset from the one or more reinforcing members by about 90 degrees.

15. The medical catheter of claim 14, wherein the distal portion comprises a deflectable region defining a longitudinal axis.

16. A medical catheter comprising:
a flexible shaft having a proximal portion, a distal portion, and a transition region between the proximal and distal portions of the shaft wherein:
the proximal portion of the shaft extends distally from the handle and is formed from a proximal shaft component wherein a first length of the proximal shaft component extends distally;
wherein distal portion of the shaft is formed from a distal shaft component wherein a second length of the distal shaft component extends proximally, wherein the distal portion comprises a deflectable region defining a longitudinal axis, wherein the deflection region is configured to assume a curved shape when a deflection force is applied, and
wherein the transition region of the shaft comprises the first length of the proximal shaft component and the second length of the distal shaft component; and
a structural element positioned within the shaft.

17. The medical catheter of claim 16, wherein the structural element is a Bowden assembly, the Bowden assembly is positioned within the proximal portion of the shaft, wherein the Bowden assembly comprises:
a pair of diametrically opposed helical Bowden cables, and a Bowden ring attached to the distal end of the Bowden cables, wherein the Bowden cables establish passageways configured for one or more steering elements extending from the proximal region to the distal region.

18. The medical catheter of claim 16, wherein the structural element is an articulation structure having a proximal end and is positioned within the distal portion of the shaft, the articulation structure comprising:
a plurality of longitudinally-arranged articulation elements, and
one or more reinforcing members extending through the articulation elements.

19. A method of producing an integrated medical catheter comprising:
providing a proximal portion of a shaft, wherein the proximal portion is formed from a proximal shaft component wherein a first length of the proximal shaft component extends distally,
providing a distal portion of a shaft, wherein the distal portion is form from a distal shaft component wherein a second length of the distal shaft component extends proximally,
performing a transition process between the proximal shaft component and the distal shaft component to create a flexible shaft,
positioning a structural element within the shaft, and
securing a handle to the proximal portion of the catheter shaft, the handle configured for manipulation by a user.

20. The method of claim 19, wherein the transition process comprises:
forming at least two recesses on the proximal shaft component using a removal process, the at least two recesses being circumferentially spaced apart and extending longitudinally along the first length of the proximal shaft component, and
disposing the distal shaft component over the proximal shaft component such that the distal shaft component fills the at least two recesses to form at least two protrusions extending longitudinally from the second length of the distal shaft component into the first length of the proximal shaft component.
